(19) United States
(12) Patent Application Publication
(10) Pub. No.: US 2011/0306531 A1
(43) Pub. Date: Dec. 15, 2011

(54) ETHER POLYSULFIDES AND POLYETHER POLYSULFIDES, THEIR PREPARATION AND USE

(76) Inventors: Yang Cheng, Midland, MI (US); Cynthia L. Rand, Sanford, MI (US); John B. Cuthbert, Midland, MI (US)

(21) Appl. No.: 13/152,314

(22) Filed: Jun. 3, 2011

Related U.S. Application Data
(60) Provisional application No. 61/353,747, filed on Jun. 11, 2010.

(51) Int. Cl. C10M 135/34; C10M 135/22; C07C 321/14; C07D 331/04; C07D 339/04; C07D 341/00

(52) U.S. Cl. 508/300; 549/36; 549/19; 568/22; 549/88; 508/570; 508/299

(57) ABSTRACT

Novel sulfur-containing compositions comprise a polyether polysulfide or an ether polysulfide. The polyether polysulfide may be based upon an alkoxylate (an alcohol or glycol-initiated compound) that is a copolymer of at least two of ethylene oxide, propylene oxide and butylene oxide. The polyether polysulfide may alternatively be based upon a homopolymer of ethylene oxide, propylene oxide or butylene oxide. The ether polysulfide may be based upon a methoxy-capped polyglycol or an alkyl ether of 1,3-dichloro-2-propanol.

This invention relates generally to novel ether polysulfides and polyether polysulfides, their preparation and their use in applications such as a lubricant or an extreme pressure (EP) additive for a metal working fluid.

Chlorinated paraffins, which constitute a major class of EP additives for metal working fluids, are under increasing regulatory pressure, especially in Europe where may are banned. Chlorinated paraffins tend to pose disposal challenges and may shorten tool life.

Pursuit of alternatives to chlorinated paraffins leads to those based upon phosphates, which tend to be limited to water-based systems, and sulfur-containing additives. Typical sulfur-containing additives include sulfurized olefins and sulfurized fatty acid esters. While such alternatives address some concerns related to chlorinated paraffins, many seek further improvements upon, or replacements for, these alternatives.

Swedish patent (SE) 120181 (B. Groth et al.) discloses a method for plasticizing rubber or a rubber-like material using a polyethylene glycol polysulfide or a derivative thereof represented by a formula as follows:

R₁—O—(CH₂—CH₂—O)ₙ—CH₂—CH₂—S_{X}—CH₂—CH₂—(O—CH₂—CH₂)ₘ—O—R₂, where x is greater than 2, m and n are integers greater than or equal to 0, and R₁ and R₂ are hydrogen or monomeric organic groups such as alkyl groups. Example compounds include (HOCH₂—CH₂—O—CH₂—CH₂)₂S₃ and (HOCH₂—CH₂—O—CH₂—CH₂)₂S₄. HO—CH₂—CH₂—O—CH₂—CH₂—O—CH₂—CH₂—S—S—CH₂—CH₂—O—CH₂—CH₂—O—CH₂—CH₂—OH

I. Afanes'ey, in Prisadki k Maslam i Topliyam, (1961), pages 58-67 discloses use of 3,6,9,16,19,22-Hexaoxa-12,13-dithiatetracosane-1,24-diol or OH—[CH₂—CH₂—O]₃—CH₂—CH₂—S—S—CH₂—CH₂—[O—CH₂—CH₂]₃—OH as an EP additive.

In some aspects, this invention is a novel sulfur-containing composition comprising a polyether polysulfide or an ether polysulfide based upon at least one of a) an alkoxylate which is an alcohol or glycol-initiated compound that is a copolymer of at least two of ethylene oxide (EO), propylene oxide (PO) and butylene oxide (BO) or, with a requirement that a linking moiety between a sulfur atom and an alkoxylate contain 1 to 5 carbon atoms, a homopolymer of PO or BO, or, with a requirement that a linking moiety between a sulfur atom and an alkoxylate contain from 3 to 5 carbon atoms, a homopolymer of EO, b) a polyglycol that is a copolymer of two or more of EO, PO and BO) or, with a requirement that a linking moiety between a sulfur atom and a polyglycol moiety contain 1 to 5 carbon atoms, a homopolymer of PO or BO, or, with a requirement that a linking moiety between a sulfur atom and an alkoxylate contain from 3 to 5 carbon atoms, a homopolymer of EO, c) a methoxy-capped polyglycol, or d) an alkyl ether of 1,3-dichloro-2-propanol, which preferably gives cyclic polysulfides.

In some aspects, such a novel sulfur-containing composition is a polyether polysulfide represented by a formula as follows:

R₁—O—(C₂H₄O)ₘ—(C₃H₆O)ₙ—(C₄H₈O)ₒ—R₂—Sₚ—R₂—(C₄H₈O)ₒ—(C₃H₆O)ₙ—(C₂H₄O)ₘ—O—R₁ where R₁ is hydrogen or an alkyl moiety containing from 1 to 18 carbon atoms, R₂ is an alkyl moiety containing from 1 to 5 carbon atoms, m is 0 or an integer within a range of from 1 to 50, n is 0 or an integer within a range of from 1 to 10, o is 0 or an integer within a range of from 1 to 20 and p is an integer within a range of from 2 to 8, provided that where m is an integer within a range of from 1 to 50, n and o cannot both be 0. Such a polyether polysulfide is at least one of a block polymer or a random polymer.

In some aspects, the polyether polysulfide is represented by a formula as follows:

R₁—O—(C₂H₄O)ₘ—R₂—Sₚ—R₂—(C₂H₄O)ₘ—O—R₁

where R₁ is hydrogen or an alkyl moiety containing from 1 to 18 carbon atoms, R₂ is an alkyl moiety containing from 3 to 5 carbon atoms, m is an integer within a range of from 1 to 50, and p is an integer within a range of from 2 to 8.

In some aspects, the novel sulfur-containing composition is an ether polysulfide represented by a formula as follows:

R₃—O—R₄

where R₃ is an linear or branched alkyl moiety containing from 3 to 18 carbon atoms, and R₄ is at least one of a sulfur-containing alicyclic moiety that has 3 carbon atoms and from 1 to 5 sulfur atoms or a sulfur-containing aliphatic moiety that has 3 carbon atoms and from 1 sulfur atom to 5 sulfur atoms. The sulfur-containing moiety, whether it is an alicyclic moiety or an aliphatic moiety, preferably contains from one sulfur atom to three sulfur atoms. The sulfur-containing aliphatic moiety may have a terminal hydrogen sulfide (—SH) moiety. The sulfur-containing aliphatic moiety may also be connected through S—S to form oligomer (2 to 8 repeat units) or polymer (more than 8 repeat units).

The novel sulfur-containing compositions described above have utility in a variety of applications including use as extreme pressure additives in lubricants or metal working fluids. A novel sulfur-containing composition as described above may also be used as a building block for a larger material, e.g. in a condensation reaction either by condensing with itself or with another molecule.

Methods to prepare the novel sulfur-containing compositions include those more fully detailed in working examples below. In general, heat a mixture of at least one sulfur source, such as a combination of one equivalent of sodium sulfide and/or sodium hydrosulfide and zero to five equivalents of sulfur like that used in Example 1 below, and a polar solvent such as ethanol under an inert atmosphere such as that provided by nitrogen to a temperature (e.g. 50° C.-100° C., with 75° C. being suitable with ethanol as a solvent) sufficient to initiate a reaction between the sulfur source and subsequently added reactants. To the heated mixture, add, with stirring, a halide reagent (0.5 equivalent to three equivalents) such as alkyl ether of 1,3-dichloro-2-propanol (decane, 2-chloro-1-(chloromethyl)ethoxy-CAS #1223394-49-7) in ethanol, 2-chloro-ethyl ethyl ether in ethanol, or 1-bromo-2-(2-methoxy-ethoxy)ethane in ethanol, and maintain the resulting mixture at that temperature for period of time sufficient to form a desired reaction product (e.g. 16 hours) and then recover the product by a procedure such as that outlined in the working examples below.

In an alternate procedure, heat a mixture of an initiator such as 3,3′-dihydroxydiphenyl disulfide and a catalyst such as 1,2-dimethylimidazole in 1,2-dimethoxyethane to a temperature sufficient to melt the initiator (e.g. 120° C. for 3,3′-dihydroxydiphenyl disulfide) then add, with stirring, an alkylene oxide (one or more of EO, PO and BO) to form a reaction mixture and maintain the reaction mixture at the temperature for a period of time (e.g. four hours) sufficient to form a desired reaction product. Recover the reaction product as detailed herein or by any other suitable means known to those skilled in the art.

One can, by selecting an appropriate polyether segment for incorporation into a polyether polysulfide, ether polysulfide or sulfur-containing material, tailor solubility of the polyether polysulfide, ether polysulfide or sulfur-containing material in a base fluid or base oil. It is known that polyethers such as polyethylene glycol or UCON™ 50-HB series polyalkylene glycols are water soluble, whereas polyethers such as polypropylene glycol are soluble in both water and some base oils such as esters, and polyalkylene glycols based on homopolymers of butylene oxide or butylene oxide-propylene oxide copolymers are miscible in Group I through Group V base oils and are insoluble in water.

In addition to affording an opportunity to tailor solubility in water or a base oil, an advantage of materials of various aspects of this invention is an ability to optimize performance properties based on needs determined by needs of a given end use application. For example, it is known that the amount of sulfur in an EP additive determines load carrying capability of the formulation containing the EP additive. EP additives with disulfides have a lower Load Wear Index (LWI) than additives based on tri- or higher polysulfides. One may, by appropriate choice of sulfur-containing raw materials, vary sulfur content of polyether polysulfides, ether polysulfides and sulfur-containing materials of various aspects of this invention and, by extension, LWI of lubricants or metal working fluids that comprise a base fluid or oil and an EP additive that is at least one of such polyether polysulfides, ether polysulfides or sulfur-containing materials. It is also known the amount of sulfur present in an EP additive affects how corrosive a lubricant or metal working fluid is to copper. Lubricants or metal working fluids that contain EP additives with disulfides tend to be less corrosive to copper than formulations that contain EP additives with tri- or higher polysulfide content. One may, by manipulating ratios of sulfur-containing raw materials to polyether, effectively control the amount of sulfur in the polyether polysulfides, ether polysulfides and sulfur-containing materials of various aspects of this invention, and thereby also control corrosiveness of lubricants or metal working fluids that comprise a base fluid or oil and an EP additive that is at least one of such polyether polysulfides, ether polysulfides or sulfur-containing materials. Other properties, such as the liquid viscosity of polyether polysulfides, ether polysulfides and sulfur-containing materials of various aspects of this invention, may be controlled by the appropriate selection of the polyether, with higher molecular weight (e.g. more than 100 Daltons) polyethers producing fluids with higher viscosities and lower molecular weight (e.g. 100 Daltons or less) polyethers producing additives with lower viscosity than said higher molecular weight polyether polysulfides, ether polysulfides and sulfur-containing materials.

Similar compositions can be derived from other well known standard sulfur chemistry involving other sulfur reagents, such as H₂S or S₂Cl₂.

### EXAMPLE

### (Ex) 1

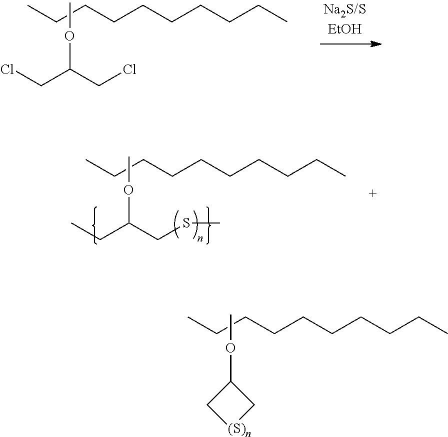

Form a mixture by charging 25 grams (g) (320.3 millimoles (mmol)) of anhydrous sodium sulfide, 30.8 g (961 mmol) of sulfur and 600 milliliters (mL) of ethanol into a 1-liter (L) 3 necked round bottom flask equipped with a stiffing bar, condenser, and addition funnel. Cover the mixture with nitrogen and heat the mixture to 75 degrees Celsius (° C.) before slowly adding 86.3 g (320.3 mmol) of decane, 2-chloro-1-(chloromethyl)ethoxy-(CAS #1223394-49-7) in 80 mL of ethanol to the flask via the addition funnel. Keep flask contents at 75° C. for 16 hours during which time sodium chloride forms as a solid. Cool flask contents to room temperature (nominally 25° C.), then filter the contents to remove solids. Concentrate filtrate in a rotary evaporator to yield a crude mixture. Dilute the crude mixture with toluene, stir it with some (10 volume % of total organic phase) 10% acetyl alcohol (AcOH) for 1 hour, and then allow it to separate into an aqueous phase and an organic phase. Wash the organic phase with aqueous sodium chloride (NaCl/H₂O) and dry it over magnesium sulfate (MgSO₄). Remove the toluene with a rotary evaporator to yield 91.7 g of dark red oil as a product. Carbon 13 nuclear magnetic resonance spectroscopy (¹³C NMR) and LC-mass spectroscopy analysis results are consistent with the product shown in the above formula in this Ex 1. The product consists primarily of di-sulfides (n=2) and trisulfides (n=3) with lesser amounts of materials possessing terminal —SH groups or oligomeric components.

### Ex 2

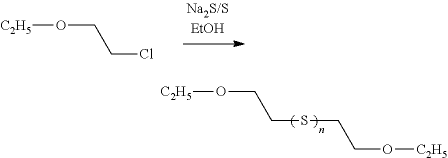

Replicate Ex 1, but change reagent and amounts thereof as follows: 15.6 g (200 mmol) of anhydrous sodium sulfide, 19.2 g (600 mmol) of sulfur, 43.4 g (400 mmol) of 2-chloro-ethyl ethyl ether and 400 ml of ethanol. Obtain 36.8 g of oil product. ¹³C NMR and LC-mass spectroscopy analysis results are consistent with the product shown in the above formula in this Ex 2. The product consists primarily of di-sulfides (n=2) and poly-sulfides (n=2 to 7.

### Ex 3

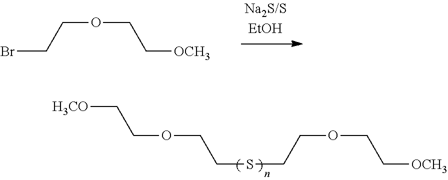

Replicate Ex 1, but change reagent and amounts thereof as follows: 1.95 g (25 mmol) of anhydrous sodium sulfide, 2.41 g (75 mmol) of sulfur, 9.15 g (50 mmol) of 1-bromo-2-(2-methoxy-ethoxy)ethane and 60 ml of ethanol. Obtain 4.34 g of oil product. ¹³C NMR and LC-mass spectroscopy analysis results are consistent with the product shown in the above formula in this Ex 3. The product consists primarily of di-sulfides (n=2) and poly-sulfides (n=2 to 7.

### Ex 4

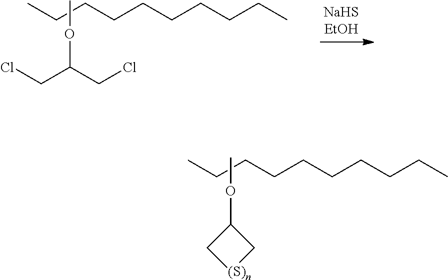

Replicate Ex 1, but change reagent and amounts thereof as follows: 14.02 g (150 mmol) of sodium hydrosulfide hydrate (˜60% of NaHS), 150 mL of ethanol, and 13.46 g (50 mmol) of decane, 2-chloro-1-(chloromethyl)ethoxy-(CAS # 1223394-49-7) in 10 mL of ethanol Obtain 11.11 g of oil product. ¹³C NMR and LC-mass spectroscopy analysis results are consistent with the product shown in the above formula in this Ex 5. The product consists primarily of mono-sulfides (n=1) and di-sulfides (n=2) and a minor fraction of the product having a terminal —SH group.

### Ex 5

For a direct alkoxylation, effect reactions using a Symyx PPR® (Parallel Pressure Reactor or PPR) setup containing 48 (6×8) small reactors. Deliver propylene oxide (PO) to the setup via an Isco syringe pump equipped with a robotically controlled needle and compressed gas microvalve. Design cell setup (reactor) layout using Library Studio® (library MFRM-306385). Dry overnight at a temperature of 125° C. a glass insert along with a removable PEEK (polyether ether ketone) stir paddle for each cell. Manually charge initiator (3,3′-dihydroxydiphenyl disulfide, 1.25 g; 5.77 mmol) and catalyst (0.125 ml of a 0.2 M solution of 1,2-dimethylimidazole in 1,2-dimethoxyethane) into the glass inserts under nitrogen. Determine weights of the glass inserts with the reaction components, then load the glass inserts along with the stir paddles into the corresponding PPR wells and seal the reactors.

Heat the whole reactor system to 120° C. to melt the initiator. Then add a calculated amount (2.32 g; 40 mmol) of propylene oxide (PO) to each reactor. Stir contents of each reactor for four hours at the temperature of 120° C., then cool the reactors to ambient (usually 25° C.) temperature before venting the reactors and purging them with nitrogen to remove residual PO. Remove the glass inserts and weigh them to calculate amount of PO consumed in the reaction. Analyze product contained in the reactors by gas chromatograph mass spectroscopy (GC/MS). The major component with 88 GC area % corresponds to the structure as shown below—the initiator with one molecule of PO at each of its sides.

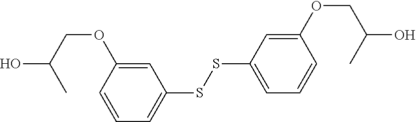

Use elemental analysis to determine sulfur content (S wt %) of CEx A (a commercial EP additive (TDPS or ditertdodecylpolysulfide, Chevron Phillips Chemical Company), Ex 1, Ex2, Ex3 and Ex 5. Exclude Ex 4 from testing as it is not soluble in base oils as noted above. Summarize sulfur content test results in Table 1 below.

Blend 5 wt % of each of Ex 1, Ex 2, Ex 3, Ex 4 and CEx A into a paraffinic base oil (AMERICAS CORE™ 100, a commercially available 100 solvent neutral paraffinic oil from ExxonMobil CEx B) and subject the blends to testing in accord with American Society for Testing and Materials (ASTM) D 2783 (Measurement of Extreme-Pressure Properties of Fluid Lubricants) to determine Load Wear Index, with a higher Load Wear Index indicating better Extreme Pressure properties, and ASTM D 4172 (Wear Preventative Characteristics of Lubricating Fluid) to determine Average Wear Scar at a loading of 40 kilograms (kg), with a lower Average Wear Scar indicating better anti-wear properties. Entries for CEx B alone serve as a control example, e.g., the base oil with no additive. Summarize testing results in Table 2 below. Two entries for CEx B, Ex 2 and Ex 1 (Table 2) show a second evaluation of each, with differences between the two evaluations being attributed to experimental error.

TABLE 1

| | Example Number | | | | |
|---|---|---|---|---|---|
| | CEx A | Ex 2 | Ex 1 | Ex 3 | Ex 4 |
| S wt % | 19.4 | 43.2 | 31.3 | 33.1 | 17.7 |

TABLE 2

| | Example Number | | | | | |
|---|---|---|---|---|---|---|
| | CEx B | CEx A | Ex 2 | Ex 1 | Ex 3 | Ex 4 |
| ¹Load | 16.1, | 40.4 | 70.4, 52.8 | 55.8, 38.1 | 66.2 | 25.1 |
| Wear | 16.8 | | | | | |
| Index | | | | | | |
| ²Average | 0.682, | 0.853 | 1.036, | 0.956, | 0.914 | 0.857 |
| Wear Scar | 0.783 | | 0.852 | 0.82 | | |
| @ 40 kg | | | | | | |

¹ASTM D2783
²ASTM D4172.

The data in Table 2 show that the products of Ex 1 through Ex 4 all improve the load wear index of the base oil (CEx B), with those of Ex 1 through 3 performing at least as well as, and in many cases better than, the commercial EP additive of CEx A). The average wear scar data for Ex 1 through Ex 4 are, in some cases better than that of CEx A, in others comparable to CEx A and in others a bit worse than CEx A. The combination of load wear index and average wear scar for Ex 1 through Ex 4 suggests that the products of those examples have utility as EP additives.

1. A polyether polysulfide represented by a formula as follows:
R₁—O—(C₂H₄O)ₘ—(C₃H₆O)ₙ—(C₄H₈O)ₒ—R₂—Sₚ—R₂—(C₄H₈O)ₒ—(C₃H₆O)ₙ—(C₂H₄O)ₘ—O—R₁
where R₁ is hydrogen or an alkyl moiety containing from 1 to 18 carbon atoms, R₂ is an alkyl moiety containing from 1 to 5 carbon atoms, m is 0 or an integer within a range of from 1 to 50, n is 0 or an integer within a range of from 1 to 10, o is 0 or an integer within a range of from 1 to 20 and p is an integer within a range of from 2 to 8, provided that where m is an integer within a range of from 1 to 50, n and o cannot both be 0.

2. A polyether polysulfide represented by a formula as follows:
R₁—O—(C₂H₄O)ₘ—R₂—Sₚ—R₂—(C₂H₄O)ₘ—O—R₁
where R₁ is hydrogen or an alkyl moiety containing from 1 to 18 carbon atoms, R₂ is an alkyl moiety containing from 3 to 5 carbon atoms, m is an integer within a range of from 1 to 50, and p is an integer within a range of from 2 to 8.

3. The polyether polysulfide of claim 1, wherein R₁ is hydrogen and said polyether polysulfide is at least one of a block polymer or a random polymer.

4. An ether polysulfide represented by a formula as follows:
R₃—O—R₄
where R₃ is an linear or branched alkyl moiety containing from 3 to 18 carbon atoms, and R₄ is at least one of a sulfur-containing alicyclic moiety that has 3 carbon atoms and from 1 to 5 sulfur atoms or a sulfur-containing aliphatic moiety that has 3 carbon atoms and from 1 sulfur atom to 5 sulfur atoms.

5. The ether polysulfide of claim 4, wherein the sulfur-containing moiety has from one sulfur atom to three sulfur atoms.

6. The ether polysulfide of claim 4, wherein the sulfur-containing aliphatic moiety has a terminal hydrogen sulfide moiety.

7. The ether polysulfide of claim 4, wherein the sulfur-containing aliphatic moiety contains at least two sulfur atoms and is connected through a bond between two adjacent sulfur atoms to form an oligomer or polymer.

8. A sulfur-containing composition comprising a polyether polysulfide or an ether polysulfide based upon at least one of a) an alkoxylate which is an alcohol or glycol-initiated compound that is a copolymer of at least two of ethylene oxide, propylene oxide and butylene oxide or, with a requirement that a linking moiety between a sulfur atom and an alkoxylate contain 1 to 5 carbon atoms, a homopolymer of propylene oxide or butylene oxide, or, with a requirement that a linking moiety between a sulfur atom and an alkoxylate contain from 3 to 5 carbon atoms, a homopolymer of ethylene oxide, b) a polyglycol that is a copolymer of two or more of ethylene oxide, propylene oxide and butylene oxide or, with a requirement that a linking moiety between a sulfur atom and a polyglycol moiety contain 1 to 5 carbon atoms, a homopolymer of propylene oxide or butylene oxide, or, with a requirement that a linking moiety between a sulfur atom and an alkoxylate contain from 3 to 5 carbon atoms, a homopolymer of ethylene oxide, c) a methoxy-capped polyglycol, or d) an alkyl ether of 1,3-dichloro-2-propanol.

9. A lubricant or metal working fluid comprising a) a base oil selected from water and Group I through Group V base oils, and b) the sulfur-containing composition of claim 8.

10. The polyether polysulfide of claim 2, wherein R₁ is hydrogen and said polyether polysulfide is at least one of a block polymer or a random polymer.
